(19) United States
(12) Patent Application Publication
(10) Pub. No.: US 2010/0217357 A1
(43) Pub. Date: Aug. 26, 2010

(54) Methods and Apparatus for Personal Care

(76) Inventors: Luiz B. DA SILVA, Danville, CA (US); T. Debuene CHANG, Danville, CA (US); Igor G. KOCHEMASOV, Nizhny Novgorod (RU)

Correspondence Address: Debuene Chang, PO Box 1156, Danville, CA, 94526, US

(21) Appl. No.: 12/708,464

(22) Filed: Feb. 18, 2010

Related U.S. Application Data
(63) Continuation-in-part of application No. 12/263,159, filed on Oct. 31, 2008.
(60) Provisional application No. 61/207,998, filed on Feb. 19, 2009.

(51) Int. Cl. A61N 5/06; B43K 5/14; A45D 44/00; A61F 7/00; A61H 1/00

(52) U.S. Cl. 607/88; 401/132; 132/212; 607/96; 601/46

(57) ABSTRACT

Methods, apparatus and systems for personal care, such as treatment of a skin or hair treatment surface, are disclosed. In some embodiments, a base device is configured to thermally modify (heat or cool) an applicator on a hand piece that can then be used to cleanse, treat and/or otherwise rejuvenate the treatment surface. The applicator can be formulated with a smooth or abrasive material that can absorb, be coated with or otherwise take up a variety of materials, such as liquids, lotions, gels or creams, which are suitable for application to the treatment surface. The base device may thermally modify the material that is then coupled to the applicator and applied to the treatment surface during treatment. Application of the applicator at the desired temperature to the treatment surface may, for example, stimulate deeper blood circulation, clear pores, diminishes or removes deep and fine wrinkles, accelerate acne clearing, treat blemished skin, decrease edema and erythema of the skin, and/or reduce puritis of various etiologies. The hand piece may comprise one or more secondary treatment elements, such as LEDs for photoactivation, electrodes for delivery of micro-currents or vibrating elements for rubbing or abraiding, in order to achieve additional activation of the treatment surface and/or of the thermally modified material coupled to the applicator, which may enhance or improve treatment.

### CROSS-REFERENCE TO RELATED APPLICATIONS

This application is a continuation-in-part of U.S. patent application Ser. No. 12/263,159, filed 31 Oct. 2008, which claims priority to U.S. Provisional Patent Appl. Ser. No. 61/002,088, filed 5 Nov. 2007, both of which are incorporated herein by reference in their entireties. Furthermore, this application claims priority to U.S. Provisional Patent Appl. Ser. No. 61/207,998, filed 19 Feb. 2009, which is incorporated herein by reference in its entirety.

### BACKGROUND OF THE INVENTION

A variety of devices and lotions are available to cleanse and rejuvenate skin. Methods of rejuvenating skin range from aggressive techniques, such as face lift surgery, laser skin resurfacing and chemical peel, to milder techniques, such as non-ablative lasers systems, RF energy skin rejuvenation systems and micro-dermabrasion. These techniques offer the potential for a younger looking appearance, the removal of wrinkles, clearing pores, reducing pitting and scarring regions, the reduction or removal of discolorations and darkened areas of the skin, and the possibility of clearing some types of undesirable skin conditions (e.g. acne, scars, and rosacea). Many of these systems are large and/or require professional treatment. Smaller, handheld devices with abrasive tips have also been previously described for use in rejuvenating skin and removing skin blemishes. See, for example, U.S. Pat. Nos. 2,712,823 and 4,378,804, both of which are incorporated herein by reference in their entireties.

### SUMMARY OF THE INVENTION

A need exists for a personal care system that can heat, cool or light activate a personal care product to effectively cleanse, rejuvenate and tone skin, hair, or a combination thereof. In some previous handheld skin rejuvenation devices, the treatment applicator and/or lotion are configured for use at room or skin temperature. The present invention provides hot or cold treatment applicators with the ability to light activate the lotion and/or to concurrently discharge micro-currents to treatment surfaces, which may stimulate deeper blood flow, reduce pore size, reduce discomfort during treatment, and/or may be used for specific targeted applications to treat specific skin conditions. Personal care apparatus, systems and methods for rejuvenating skin, treating skin conditions and skin lesions such as acne, removing fine wrinkles, preventing discoloration and blemishes, reducing edema and swelling, calming dermatitis and skin irritations, toning skin and/or clearing skin of other inflammatory and erythematous processes are disclosed.

A hand held device and method for safely exfoliating, cleansing and/or toning skin with minimal discomfort or pain, while leaving a moisturizing and protective layer for longer-term treatment, is also disclosed. Furthermore, a device and method for heating, cooling and/or light activating personal care products or materials to increase or accelerate efficacy is disclosed. The personal care products may include soaps, creams, wipes or towelettes, washes, shampoos, conditioners, sprays, lotions, balms, enamels, tonics, mousses or combinations thereof. The personal care products can be applied, and designed for application, on the face, hands, body, hair, eyes, lips, or combinations thereof.

A thermal device that can cool and/or heat an encapsulated personal care product pack, and a treatment hand piece that can also light the personal care product pack, are disclosed. The encapsulated personal care product pack can be placed within the thermal device and cooled or heated to a desired temperature. The encapsulated pack can be substantially thermally insulated. The encapsulated pack can have pack contents, such as a fluid or gel or cream or lotion. The encapsulated pack can be a cartridge, blister pack, ampule, or other openable and exchangeable container. The encapsulated pack can have a removable protective cover that can contain the pack contents in the pack.

The pack is configured for coupling to a thermal device, such that a surface of the pack comes into thermal contact with a thermoelectric surface of the thermal device for altering or modifying a temperature of the pack and/or the pack's contents, i.e. for cooling or heating the pack and/or its contents. In one embodiment, the thermal device can cool the pack to a low enough temperature to freeze the pack's contents. In one embodiment, the thermal device can heat the pack to a temperature sufficient to vaporize the pack's contents. In one embodiment, the thermal device can heat and/or cool the pack to a desired temperature between freezing and vaporization of the pack's contents.

The treatment hand piece or a portion thereof, such as an applicator of the hand piece, may be placed into the pack to at least partially absorb or otherwise take up the pack's contents. By thermal conduction, the thermal device, the pack and/or the pack's contents optionally may modify the temperature of the hand piece applicator. It should be understood that the thermal device may be used to heat or cool the pack and/or its contents prior to, during or after placement of the hand piece applicator into the pack.

When the pack and/or its contents reach a desired temperature, the user may be notified, e.g., via a notification element such as an emitted sound or a light indicator. A temperature sensor, such as a thermocouple, may be provided for measuring the temperature of the pack and/or its contents. The hand piece applicator may be removed from the thermal device with at least some of the thermally modified pack contents disposed thereon, and may be applied to skin or hair. The user may apply the hand piece and thermally modified pack contents to his or her own skin or hair, and/or may apply the hand piece and thermally modified pack contents to another person's skin or hair.

In one embodiment, the hand piece contains light emitting diodes (LEDs) that can be turned on to light activate the contents of the pack in preparation for skin or hair application. Since the LEDs are disposed in the hand piece, activation of the pack contents, e.g. lotion, may continue while the hand piece is applied to skin or hair during treatment. Light activation of some lotions or other pack content materials may produce oxygen radicals, which may be effective in killing bacteria.

The hand piece can be manipulated to apply the pack contents to the skin or hair. In one embodiment, the hand piece includes a motor that vibrates the hand piece and its applicator to increase abrasion. Rubbing of skin with the hand piece applicator, e.g. by vibrating the hand piece, may exfoliate the skin, for example, by removing the top layers of the stratum corneum and cleansing the skin of dirt, grease, and debris. In one embodiment, the hand piece also may include a thermal element to maintain the temperature of the applicator at the desired temperature during treatment. In one embodiment, the hand piece and the thermal device may be integrated into a single device.

Depending on the needs of the user, either a hot or cold temperature of the treatment applicator and/or the pack contents may be chosen. For instance, a hot temperature of the applicator may stimulate blood flow in the dermis. A cold temperature of the applicator and/or the pack also may stimulate blood flow in the dermis as a reflexive response to cold in the upper surface layers of the epidermis. Furthermore, a cold temperature may reduce inflammation and edema with minimal discomfort or pain, even when more aggressive dermabrasion is utilized.

The temperature of the applicator and/or the pack contents optionally may be altered during treatment in order to perform a multiple step thermal treatment. In one embodiment, the hand piece may include a control element, such as a toggle switch or button, to change temperatures of the applicator as desired. In one embodiment, the hand piece comprises an automated treatment cycle with instructions that are programmed into the hand piece.

In some embodiments, the hand piece may include an electro-generating unit that produces micro-currents, which may be passed through the applicator to a treatment surface such as skin or hair. Micro-currents may increase tone and/or may stimulate structural and protein content changes within the epidermal layers of the skin.

The pack contents may include drugs or pharmaceuticals (e.g., benzoyl peroxide, salicylic acid, glycolic acid, antiseptic, antibiotics, steroids, anti-inflammatories and combinations thereof) and/or cosmetics (e.g., emollients, moisturizers, botanicals, fragrances, vitamins, anti-oxidants and combinations thereof) that are, for example, effective at treating a variety of skin or hair conditions. Through the use of the applicator, these pharmaceuticals and cosmetics can be left on the treatment surface (e.g. skin or hair) after application to the surface, for example to sooth, moisturize, and protect the skin for long term treatment afterwards.

The hand piece optionally can accommodate a variety of applicators that attach directly to the hand piece. Applicators may have one surface that connects firmly to the hand piece and another surface that holds at least some of the pack contents and comes into contact with skin or hair. The side of the applicator that contacts skin or hair can include, for example, foam, cotton pad, bristles, abrasive ceramic and combinations thereof. The applicator also may comprise an electrically conductive surface that can be used to apply micro-currents and electrical stimulation to the skin.

Before the hand piece applies the pack's contents to skin or hair, the thermal device may thermally alter or modify, e.g. heat or cool, the pack's contents. The thermal device optionally may be programmed or may comprise instructions for thermally modifying the pack's contents at a user-selected time that is convenient for personal use. In one embodiment, thermal modification (cooling or heating) of the pack and its contents may be completed in less than about 20 minutes. In one embodiment, such thermal modification takes less than about 5 minutes. In one embodiment, the encapsulated pack and/or its contents can be placed within the thermal device and cooled or heated to a desired temperature in a range of about −40° C. to about 100° C. In one embodiment, the pack and/or its contents may be cooled or heated to a desired temperature in a range of about −20° C. to about 80° C. In one embodiment, the pack and/or its contents may be cooled or heated to a desired temperature that is at least 10 degrees different (i.e. above or below) from ambient temperature.

Before, during or after thermal modification of the encapsulated pack and/or its contents to the desired temperature, all or a portion of the pack and/or its contents may be coupled to the hand piece, e.g. disposed on the application. In one embodiment, the pack may be removed from the thermal device prior to coupling of the pack and/or its contents to the hand piece. In another embodiment, the pack and/or its contents may be disposed within the thermal device when coupled to the hand piece. A protective cover on the pack may be removed, or the pack otherwise may be opened, before, concurrent with, or subsequent to coupling (e.g., attachment or insertion) of the pack and/or its contents to the hand piece.

The hand piece and/or its applicator may be used to apply the thermally modified (heated or cooled) pack and/or pack contents to a treatment surface, such as skin or hair. In some embodiments, an LED light can activate the pack contents before, during and/or after application of the pack contents to the treatment surface. In some embodiments, the hand piece and/or its applicator may include electrodes configured to generate micro-currents for stimulating the treatment surface and/or increasing treatment penetration.

Rubbing of skin with the hand piece applicator and/or thermally modified pack/pack contents may abrade or exfoliate the skin, for example, may remove the top layers of the stratum corneum, and/or may cleanse the skin of dirt, grease, and debris. Abrasion or exfoliation may encourage easier and/or more effective absorption of pharmaceuticals and/or cosmetics by the skin. Furthermore, abrasion or exfoliation may simultaneously leave a surface layer of protective or soothing moisturizer or emollient. The pack optionally may comprise an abrasive and/or fluid-absorbing layer on its surface that is configured for removal of upper keratinized cell layers from the stratum corneum of the epidermis.

When thermal modification of the pack and/or its contents comprises cooling of the pack/pack contents, the cooled pack/pack contents may have a clearing or cleansing effect on a treatment surface and may provide anti-inflammatory and/or analgesic effects. When thermal modification comprises heating, the heated pack/pack contents may stimulate blood flow and/or may open pores to expose dirt, debris or bacteria to deeper cleansing. Warm cleansing also may facilitate enhanced delivery of humoral and blood factors that clear lesions or resolve edema and erythema.

The heated or cooled pack contents (which may, for example, comprise a fluid, gel, lotion, cream or combination thereof) may include drugs or pharmaceuticals (e.g., benzoyl peroxide, salicylic acid, glycolic acid, antiseptic, antibiotics, steroids, anti-inflammatories and combinations thereof) and/or cosmetics (e.g., emollients, moisturizers, botanicals, fragrances, vitamins, anti-oxidants and combinations thereof) that may be effective at treating a variety of skin conditions. Through the use of the applicator, these pharmaceuticals and cosmetics may be left on the treatment surface post-treatment for soothing moisturization and protection and/or for long-term treatment post-application.

The thermal device may have multiple thermal functions. For example, the thermal device may thermally modify, e.g., cool and/or heat, the personal care product pack. The personal care product pack optionally can be coded or keyed, such that the thermal device may automatically determine the final desired temperature and may automatically thermally modify the product pack and/or its contents the desired final temperature. The user optionally may manually select the operating mode or temperature of the thermal device, for example, through a control element, such as a switch or button.

### BRIEF DESCRIPTION OF THE DRAWINGS

FIG. 1 is a schematic view of an embodiment of a personal care apparatus and system of the present invention.

FIG. 2 is a sectional view of the apparatus and system of FIG. 1 with the hand piece coupled to the thermal device.

FIG. 3 is a detail sectional view of the hand piece with an applicator attached.

FIG. 4 is a schematic view of a method in accordance with the present invention for treating a treatment surface, illustratively a human face.

FIG. 5 is a detail sectional view of an embodiment of a pack with flowable material for use with apparatus and methods of the present invention.

FIG. 6 is a block diagram of an embodiment of the present invention.

### DETAILED DESCRIPTION

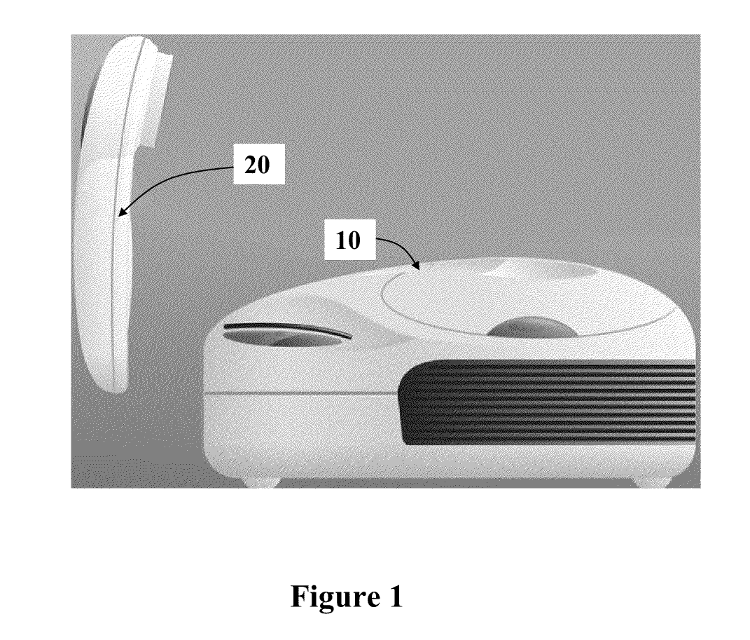

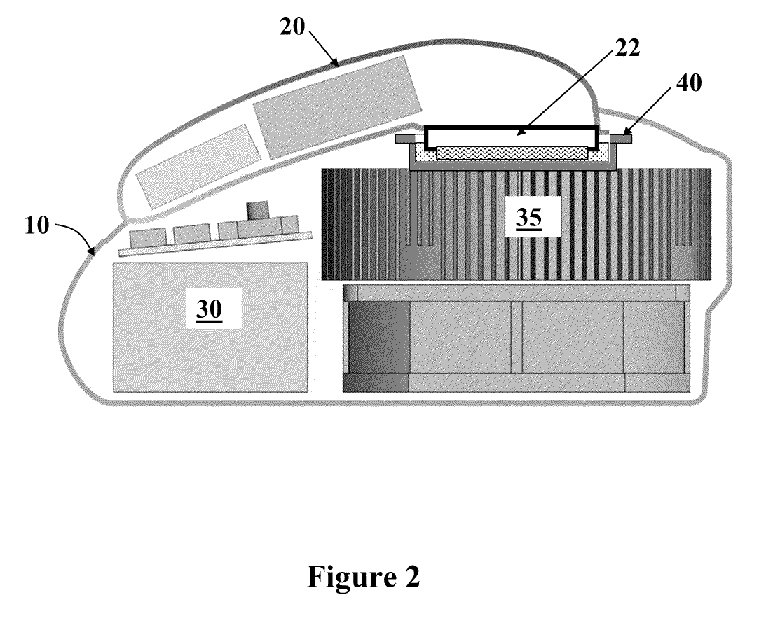

Personal care skin or hair treatment systems, apparatus and methods that can be safely used by adolescents and adults to cleanse, rejuvenate and/or treat skin conditions such as acne, blemished skin, dermatitis including eczema, hives, or other inflammatory and/or edematous lesions such as rosacea or fine wrinkles, are described herein. As seen in FIGS. 1 and 2, a personal care treatment system may comprise a base thermal device 10 and a hand piece 20. The base thermal device 10 is configured to thermally modify, i.e. heat and/or cool, an encapsulated personal care product pack 40 and/or its contents, which may, for example, comprise a fluid, gel, cream, lotion, pharmaceutical and/or cosmetic. The hand piece 20 may be detachably coupled to the base device 10 to transfer the pack 40 and/or its contents to the hand piece for application to a treatment surface, such as hair or skin.

The base thermal device 10 preferably is AC powered, but in some embodiments may be DC powered using batteries. As seen in FIG. 2, the base thermal device 10 comprises a power supply and control electronics 30 that are configured to control and modify the temperature of (i.e. heat and/or cool) thermoelectric module 35, as desired. Module 35 is in thermal communication with pack 40 prior to treatment for directly and/or indirectly modifying the temperature of the pack 40 and/or its contents. The base device 10 may accept user input to control activation (thermal modification) of the pack 40.

In some embodiments, the hand piece 20 may be a passive element without electrical components, such as heaters, motor-driven vibrators/agitators/abraiders or micro-current electrodes. In some embodiments, the hand piece 20 is electrically powered and comprises one or more of such electrical components. When powered, the hand piece 20 preferably is battery powered. The hand piece optionally may comprise rechargeable batteries that are recharged when the hand piece is detachably coupled to the base device 10. It should be understood that, while the hand piece preferably is battery powered, in some embodiments it may be DC powered.

The hand piece 20 may comprise an applicator 22 that may be permanently or temporarily attached to the hand piece 20 and that may be brought into contact with pack 40 and/or its contents when the hand piece 20 is detachably coupled to the base device 10. In some embodiments, the hand piece may be configured for use with a variety of applicators 22, which may allow users to select a desired form or style of treatment and/or which may be suited for use with a variety of different packs/pack contents. The applicator may be configured to absorb or otherwise take up all or a portion of the thermally modified pack and/or pack contents for application to a treatment surface, such as hair or skin.

The thermoelectric module 35 allows the pack 40 and/or its contents to be cooled or heated during activation. In one embodiment, thermal modification (cooling or heating) of the pack/pack contents may be completed in less than about 20 minutes. In one embodiment, such thermal modification takes less than about 5 minutes. In one embodiment, the encapsulated pack and/or its contents can cooled or heated to a desired temperature in a range of about −40° C. to about 100° C. In one embodiment, the pack and/or its contents may be cooled or heated to a desired temperature in a range of about −20° C. to about 80° C. In one embodiment, the pack and/or its contents may be cooled or heated to a desired temperature that is at least 10 degrees different (i.e. above or below) from ambient temperature. Cooling below 0° C. may allow water-based pack contents to be frozen. When heating, the maximum temperature optionally may be less than about 70° C. to reduce a risk of burns. In one embodiment the pack 40 is keyed so that the desired activation temperature is automatically determined by the base device 10.

The personal care pack 40 can be cooled or heated by the base device 10 before the treatment. Cooling may be used, for example, to reduce pain during aggressive cleansing, for skin tightening due to skin cooling that reduces pore size, to stimulate deeper blood circulation in the dermis to help carry humoral, immune and/or blood factors to affected regions to speed healing, and combinations thereof. Heating the pack 40 may, for example, increase blood circulation, accelerate chemical and enzymatic reactions, increase penetration of personal care product through the skin, open pores to expose dirt, debris and/or bacteria to deeper cleansing, and combinations thereof. The efficacy of personal care products can be directly increased by the elevated temperature, which can accelerate chemical reactions.

In one embodiment, the base device 10 comprises one or more LEDs that surround or are otherwise positioned relative to the pack 40 to allow direct photo activation of the pack contents. In one embodiment, the hand piece 20 and/or the applicator 22 may comprise one or more LEDs for such direct photo activation of the pack contents.

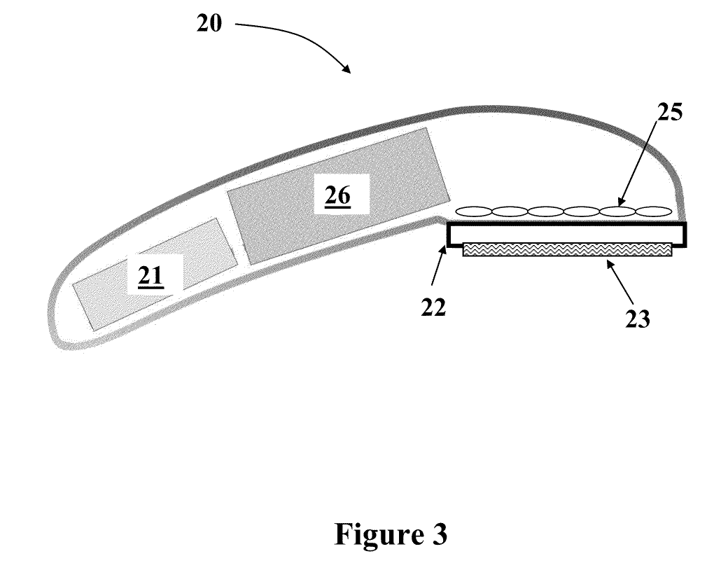

FIG. 3 illustrates a sectional view of one embodiment of the hand piece 20 with an applicator 22 attached. The applicator has a treatment layer 23. In FIG. 3, the treatment layer illustratively comprises a foam layer that can absorb pack contents, such as lotion, gel or cream, when placed into the pack 40. Additional and alternative treatment layers are discussed in more detail below.

The hand piece may include batteries 21 and control electronics. In one embodiment, the hand piece 20 includes a vibration/agitation/abrasion motor 26. The hand piece 22 additionally or alternatively may include light emitting diodes (LEDs) 25 that may be used to heat or light activate the pack contents disposed on or within the applicator 22. LED emission can be a single color in the range extending from UV to infrared. Alternatively, the LEDs can be a combination of multiple colors or may be white LEDs.

In one embodiment, the hand piece 20 and base device 10 may communicate, either through direct electrical contact when the hand piece is in its normal cradle position in contact with the base device (i.e. is detachably coupled to the base device) or through wireless communication. In this embodiment, the user may control (i.e. turn on or off) the LEDs 25 and/or the motor 26 by pressing control buttons on the base device 10. One or more buttons on the hand piece 20 may be provided for controlling the motor and/or LEDs when the hand piece is removed from the base device.

A variety of applicators 22 may be used with hand piece 20, each having a connecting side that temporarily or permanently attaches to the hand piece and a treatment layer 23. The treatment layer 23 can be soft (e.g., tissue, cloth, foam), hard, smooth, abrasive (structured plastic, porous ceramic, porous metal), or a combination thereof. The hand piece can include electrical contacts that make contact with the applicator 22 to power active treatment layers 23. For example, the treatment layer may be an electrically conductive surface with multiple electrodes to enable electrical stimulation of skin. The applicator 22 also may include a resistive heater to allow the treatment layer 23 to stay warm during the treatment procedure. The applicator 22 also may include a thermoelectric module to allow the treatment layer 23 to be cooled during the treatment procedure. In some embodiments, a control element, such as button or switch, on the hand piece 20 may be used to control the temperature of the treatment layer 23.

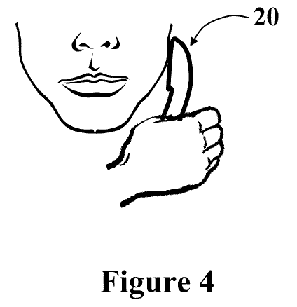

FIG. 4 shows how the activated hand piece can be used to treat a treatment surface, illustratively skin on a person's face. The hand piece 20 can be placed on the skin to treat. For example, the treatment layer 23 of the applicator 22 may transfer all or a portion of the thermally modified contents of the pack 40 to the treatment surface to achieve treatment.

The hand piece 20 can rub or abraid the treatment surface during treatment. For example, the hand piece can be manually powered by the user to rub the applicator 22 on the treatment surface by moving the hand piece 20 in a circular or back and forth pattern over the surface during treatment. Additionally or alternatively, a powered rotational/vibrational or other element of hand piece 20, such as motor 26, may be used to rotate and/or vibrate the applicator 22 to cleanse and apply the contents of pack 40 to the treatment surface.

After treatment, the applicator 22 optionally may be cleaned and placed in the base device 10 or elsewhere to dry. Alternatively, the applicator may be disposable, and a different applicator may be used for subsequent treatment. Subsequent treatment with the same or a different applicator 22 may commence immediately after a first treatment or at a later time. Subsequent treatment optionally may utilize another new and exchangeable pack 40 that can be placed in the base device 10 for thermal modification of the pack contents, as well as transfer of the thermally modified pack contents to the hand piece 20 and its applicator 22. Subsequent treatments may utilize one or more packs 40 comprising the same or different pack contents.

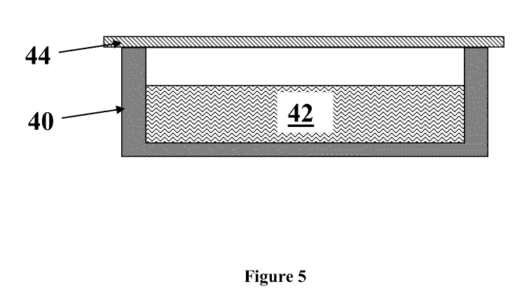

FIG. 5 provides a sectional view of an embodiment of a personal care pack 40. In FIG. 5, the pack 40 includes an outer plastic shell that contains the pack's contents, a flowable material or fluid 42, and a sealing layer 44. The pack 40 may be disposable or may be a reusable pack that can be refilled with fluid 42. The fluid 42 may comprise, for example, a gel, a cream, a liquids with or without pharmaceuticals and/or cosmetics (e.g., water, anti-oxidants, antibiotics, anti-inflammatories, moisturizers, emollients, vitamins, fragrance, cleansing and abrasive elements, and combinations thereof), and combinations thereof. The bottom surface of the pack 40 may be flat and smooth to improve thermal contact with the thermoelectric module 35 of base device 10. The sealing layer 44 optionally may be partially or completely removed before, during or after thermal modification of the fluid 42 for transferring at least a portion of the fluid to the applicator 22 of hand piece 20.

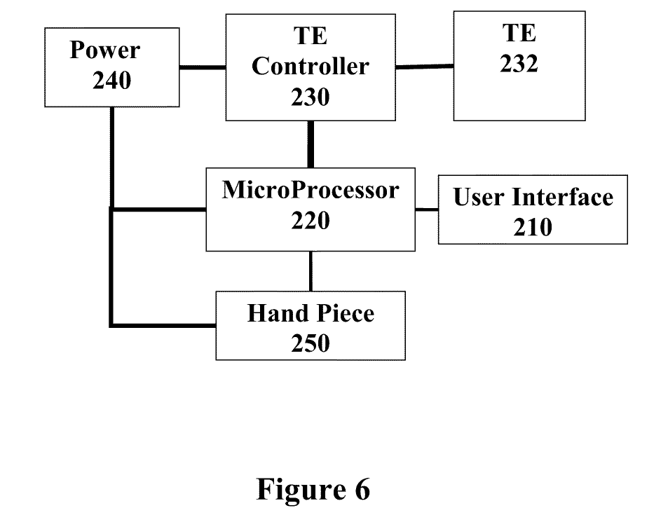

FIG. 6 provides a block diagram showing elements of an embodiment of personal care system in accordance with the present invention. A power source 240, which can be a wall plug batteries, or a combination thereof, can power the thermoelectric (TE) controller 230, as well as the thermoelectric module 232 that is controlled thereby, the microprocessor 220, the user interface 210, and (when electrically powered) the hand piece 250. The microprocessor 220 can accept user input (e.g. on/off, desired temperature, time to turn on and prepare) from the user interface 210 to control system operation. User interface 210 can include, for example, push buttons, LCDs, LEDs and buzzers. When the hand piece 250 is placed on the base device the microprocessor can communicate with the hand piece and control operation. When the hand piece 250 is placed on the base device it can also connect to the base device power and recharge the hand piece batteries.

In some embodiments, a method for using a personal care system or apparatus in accordance with the present invention may comprise one or more of the following steps: 1) Selecting a personal care pack with desired contents, e.g., desired fluid, lotion, gel or cream contents. 2) Removing a protective layer from the pack and placing the pack inside the base thermal device. 3) Placing the hand piece with the desired applicator on the base device cradle such that the applicator fits inside the pack and can absorb, be coated with or otherwise take up the contents, e.g. fluid contents, of the pack (i.e., detachably coupling the hand piece to the base device). 4) Selecting and initiating desired activation (cooling, heating, light), e.g., by manipulating a control element, such as button or switch. 5) Waiting for base device to signal that activation is complete. 6) Removing the hand piece from the base device and putting the applicator on a surface to be treated, such as skin or hair. 7) If desired, activating a secondary treatment element or aspect of the hand piece, such as vibrator/agitator/abraider, LEDs and/or micro-current electrodes for additional activation of the pack contents during treatment.

Skin improvement effects by the methods of using the system or apparatus can include, for example, cooling at the surface and allowing the energy to cool the upper layer of the dermis to achieve controlled changes to the collagen in the upper dermal layer. The system may temporarily tighten the skin from the cold and reduce the pore size of skin for a smoother texture of the skin. Cold treatment may potentiate the development of skin protein layers, such as collagen and elastin, among others, and decrease inflammation and edema. Alternatively, the system may, by heating the skin surface, induce controlled changes to the collagen in the upper dermal layer. The system may, by temporarily dilating skin pores with heat, allow deeper cleaning of dirt, debris, oils, contaminants, and bacteria. The system may deliver a controlled amount of mechanical abrasion to remove upper layers of the stratum cornium of the epidermis, resulting in easier access to pores and mechanical cleansing and debridement of dirt, contaminants, and excess sebum and bacteria. The system may allow more direct delivery of pharmaceuticals and cosmetics into deeper layers of skin for effect and treatment once the cold mechanical abrasion of the upper layers is accomplished. The system can decrease edema and erythema of the skin, thereby reducing post inflammatory hyper pigmentation preventing discoloration, uneven coloration, and/or blotching of the skin. The system may deliver LED light activated pharmaceuticals and cosmetics onto the skin surface to improve or treat skin conditions. Micro-currents may be applied to the treatment surface by the hand piece/applicator, which may improve skin and muscle tone to help reduce wrinkles.

The devices described herein are suitable for use in cleansing skin, rejuvenating skin and treatment of various skin conditions and lesions. These devices offer the potential for younger looking appearance; remove wrinkles, clearing of pores, reducing inflammation and edema, and the possibility of clearing some types of undesirable skin conditions (e.g. acne, rosacea, eczema, and other types of dermatitis).

The devices described herein are suitable for use in the treatment of various skin conditions and lesions. Examples of such skin conditions and lesions are provided herein, but it should be understood that such conditions and lesions are not limited to the conditions and lesions described herein. Bacterial infections lead to common inflammatory and edematous lesions such as acne and pimples. Other dermatitis lesions are caused by irritants, which may be introduced as a result of bug bites or by exposure to other natural or man-made skin irritants. Eczema can be widespread or localized on the skin and is consistent with inflammatory lesions. Some infections (either bacterial—i.e. staphylococcus. Streptococcus, Pseudomonasor fungal—i.e. Candida in difficult to access areas such as paronychial infections and under the nail bed) might be treated with pharmaceutical instilled dermabrasion

Other skin conditions include pustular eruptions, localized abscessed formation and local inflammatory conditions of the dermis and epidermis. One of the most common afflictions of this type is lesions caused by the condition known as acne vulgaris. Acne vulgaris is associated with the Gram-positive anaerobic bacterium, Propionibacterium acnes.

Eczema is a form of inflammatory lesions which can be localized or widespread and may be a response to known or unknown irritants. Eczema is puritic and irritating. Long-term, chronic inflammation and edema can lead to post inflammatory hyper pigmentation and discoloration of the skin with unsightly, uneven blotchiness.

Methods and devices for the treatment of skin conditions and skin lesions involving the application of mechanical abrasion and cooling and/or gentle heating to the infected or irritated tissue are disclosed. The system alone or in combination with other pharmaceuticals (e.g. antifungal, antiviral) can be used to treat skin lesions caused by bacterial, fungal, or viral infections. A skin condition or skin lesion that can be treated according to the methods disclosed herein is any infected or irritated tissue that can be effectively treated by the application of cleansing, cooling and/or heating.

For the purposes of the present invention, “treating” a skin lesion or condition includes, but is not limited to, slowing, halting or even reversing the development of the skin lesion or condition and/or reducing the lesion's or condition's healing time. Therapeutic benefit can be achieved by eradication or amelioration of the underlying disorder being treated, e.g., eradication or amelioration of the acne, and/or eradication or amelioration of one or more of the physiological symptoms associated with the condition being treated, notwithstanding that the patient may still be afflicted with the underlying disorder. The terms “subject” and “animal subject” used herein typically refer to a human, but could also include other suitable animals.

The products used with the system can also be combined with topical gels or creams to improve treatment of skin lesions, by furthering the treatment effects of the device. For example, for acne, topical gel with benzoyl peroxide, antibiotics, anti-inflammatories, such as salicylic acid can be applied after treatment with the device. For improvement of fine wrinkles, topical lotions and gels can be applied after micro-abrasion which would have greater facility in being absorbed for maximal effect. Examples include glycolic acid, moisturizing emollients, and anti-oxidants such as green tea. For eczema, anti-inflammatories and pharmaceuticals can be applied directly afterwards for quicker and easier absorption into the skin for maximal effect.

The system, methods and apparatus of the present invention can be used to improve drug delivery by applying drugs and creams to the skin before treatment. This may be in the form of a cleanser or micro-abrasion element to be worked into the skin surface by the device. It can also be micro-encapsulated pharmaceuticals or cosmetics that can be activated by the cold and mechanical abrasion of the device.

All patents and patent applications mentioned in this specification are herein incorporated by reference in their entireties. The above descriptions and illustrations are only by way of example and are not to be taken as limiting the invention in any manner. Numerous variations, changes, and substitutions can occur to those skilled in the art without departing from the invention. It should be understood that variations of the elements described herein may be employed. It is intended that the following claims define the scope of the invention and that methods and structures within the scope of these claims and their equivalents be covered thereby.

We claim:

1. Apparatus for personal care, the apparatus comprising:
a base device having a thermoelectric module;
a hand piece with an applicator; and
a flowable material configured for application to a skin or hair treatment surface,
wherein the thermoelectric module is configured to modify a temperature of the flowable material when the flowable material is in thermal communication with the thermoelectric module, and
wherein the hand piece applicator is configured to apply the flowable material to the treatment surface after the temperature of the flowable material has been modified by the thermoelectric module.

2. The apparatus of claim 1 wherein the base device further comprises a power supply and a control element coupled to the thermoelectric module for dynamically controlling a temperature of the thermoelectric module.

3. The apparatus of claim 1, wherein the hand piece applicator further is configured to transfer the flowable material from the base device to the treatment surface.

4. The apparatus of claim 1, wherein the hand piece applicator comprises an applicator chosen from the group consisting of soft applicators, tissue applicators, cloth applicators, foam applicators, cotton applicators, sponge applicators, paper applicators, electrically conductive applicators, electrically insulating applicators, hard applicators, smooth applicators, abrasive applicators, structured applicators, plastic applicators, ceramic applicators, metal applicators, disposable applicators, interchangeable applicators, replaceable applicators, permanent applicators, temporary applicators, porous applicators, non-porous applicators, and combination thereof.

5. The apparatus of claim 1, wherein the flowable material comprises a material chosen from the group consisting of fluids, foams, gels, liquids, lotions, creams, drugs, soaps, wipes, towelettes, washes, shampoos, conditioners, sprays, lotions, balms, enamels, tonics, mousses, pharmaceuticals, cosmetics, benzoyl peroxide, salicylic acid, glycolic acid, antiseptic, antibiotics, steroids, anti-inflammatories, emollients, moisturizers, botanicals, fragrances, vitamins, anti-oxidants, and combinations thereof.

6. The apparatus of claim 1 further comprising a pack, wherein the flowable material is contained within the pack while the flowable material is in thermal communication with the thermoelectric module.

7. The apparatus of claim 6, wherein the pack comprises a pack chosen from the group consisting of cartridges, blister packs, ampules, open-able packs, disposable packs, replaceable packs, reusable packs, refillable packs, sealable packs, and combinations thereof.

8. The apparatus of claim 6, wherein the hand piece applicator is configured for placement within the pack for transferring at least a portion of the flowable material to the applicator.

9. The apparatus of claim 6, wherein the pack comprises a sealing layer for containing the flowable material within the pack.

10. The apparatus of claim 9, wherein the sealing layer is configured for removal or puncture when the hand piece applicator is placed within the pack.

11. The apparatus of claim 1, wherein the hand piece further comprises a secondary treatment element.

12. The apparatus of claim 11, wherein the secondary treatment element comprises at least one light emitting diode configured to photo activate the flowable material before or during application of the flowable material to the treatment surface.

13. The apparatus of claim 11, wherein the secondary treatment element comprises a mechanism configured to vibrate or rotate the applicator while the applicator is in contact with the treatment surface.

14. The apparatus of claim 11, wherein the secondary treatment element comprises a mechanism configured to apply micro-currents to the treatment surface.

15. The apparatus of claim 11, wherein the secondary treatment element comprises a mechanism for measuring or altering a temperature of the flowable material before or during application of the flowable material to the treatment surface.

16. The apparatus of claim 11, wherein the hand piece further comprises a power supply and a control element coupled to the secondary treatment element for dynamically controlling the secondary treatment element.

17. The apparatus of claim 16, wherein the power supply comprises batteries.

18. The apparatus of claim 17, wherein the batteries comprise rechargeable batteries.

19. The apparatus of claim 18, wherein the base device is configured to recharge the rechargeable batteries while the hand piece is detachably coupled to the base device.

20. The apparatus of claim 1, wherein the base device and the hand piece are configured for communication with one another.

21. The apparatus of claim 1, wherein the applicator has a first surface configured for connecting the applicator to the hand piece and a second surface configured to contact the treatment surface.

22. A method for personal care, the method comprising:
modifying a temperature of a flowable material to a desired temperature with a thermoelectric device;
transferring the temperature modified flowable material from the thermoelectric device to a skin or hair treatment surface with a hand held applicator; and
rubbing the treatment surface with the applicator to apply the temperature modified flowable material to the treatment surface, thereby treating a condition of the treatment surface.

23. The method of claim 22, wherein modifying the temperature of and applying the flowable material further comprises modifying a temperature of and applying a flowable material chosen from the group consisting of fluids, foams, gels, liquids, lotions, creams, drugs, soaps, wipes, towelettes, washes, shampoos, conditioners, sprays, lotions, balms, enamels, tonics, mousses, pharmaceuticals, cosmetics, benzoyl peroxide, salicylic acid, glycolic acid, antiseptic, antibiotics, steroids, anti-inflammatories, emollients, moisturizers, botanicals, fragrances, vitamins, anti-oxidants, and combinations thereof.

24. The method of claim 22, wherein modifying the temperature of the flowable material to the desired temperature further comprises modifying a temperature of the applicator to the desired temperature.

25. The method of claim 22 further comprising photo-activating the temperature modified flowable material.

26. The method of claim 25, wherein photo-activating the temperature modified flowable material further comprises photo-activating the flowable material with a light emitting diode.

27. The method of claim 22, wherein rubbing the treatment surface with the applicator to apply the temperature modified flowable material to the treatment surface further comprises modifying a temperature of the treatment surface.

28. The method of claim 22, wherein rubbing the treatment surface with the applicator to apply the temperature modified flowable material to the treatment surface further comprises abraiding the treatment surface.

29. The method of claim 22 further comprising delivering micro-currents through the applicator to the treatment surface.

30. The method of claim 22, wherein modifying the temperature of the flowable material to the desired temperature further comprises modifying the temperature to a desired temperature in the range of about −40° C. to about 100° C.

31. The method of claim 22, wherein modifying the temperature of the flowable material to the desired temperature further comprises modifying the temperature to a desired temperature in the range of about −20° C. to about 80° C.

32. The method of claim 22, wherein modifying the temperature of the flowable material to the desired temperature further comprises freezing or vaporizing the material.

33. The method of claim 22, wherein modifying the temperature of the flowable material to the desired temperature further comprises modifying the temperature to a desired temperature of less than about 70° C. to reduce a risk of burns.

34. A method for personal care, the method comprising:
modifying a temperature of a flowable material to a desired temperature;
photo-activating the flowable material; and
applying the temperature modified and photo-activated flowable material to a treatment surface, thereby treating a condition of the treatment surface.
